(19) United States
(12) Patent Application Publication
(10) Pub. No.: US 2003/0074141 A1
(43) Pub. Date: Apr. 17, 2003

(54) Meta-models for predicting blood brain barrier penetration based on simple physicochemical descriptors

(75) Inventor: Stuart J. Russell, San Francisco, CA (US)

Correspondence Address: BEYER WEAVER & THOMAS LLP, P.O. BOX 778, BERKELEY, CA, 94704-0778, US

(73) Assignee: Camitro Corporation

(21) Appl. No.: 09/978,372

(22) Filed: Oct. 15, 2001

(51) Int. Cl. G06F 19/00; G01N 33/48; G01N 33/50; G01N 31/00

(52) U.S. Cl. 702/19; 702/22

(57) ABSTRACT

Descriptor based models, employing at least two descriptors, predict activities of compounds under consideration. Those activities may be biology-based activities such as the ability of the compound to cross the blood brain barrier. In one example, the model predicts a compound's solubility, its ability to be absorbed in the intestine, and its ability to cross the blood brain barrier. The descriptors of interest are typically physicochemical properties of the whole molecule. Examples include a log P or log D, molecular weight or related size-based descriptors, the number of hydrogen bond donors and/or hydrogen bond acceptors, formal charge, lipophilicity, and the like.

### BACKGROUND OF THE INVENTION

This invention pertains to models that predict chemical properties relevant to medicinal chemists using relatively simple molecular descriptors. For example, the invention pertains to models that predict blood brain barrier penetration from the simple descriptors.

New drug discovery is an expensive and highly time-consuming process. Much of the expense is a result of inability to quickly and easily determine whether a particular drug candidate (i.e., an organic chemical compound) will be properly absorbed, distributed, metabolized, and excreted when administered to human patients.

Absorption, for example, is highly relevant to medicinal chemists. If a particular compound cannot be easily absorbed within the intestine, then that compound will not be suitable for oral administration. Either the compound will have to be abandoned or other routes of administration will have to be considered.

If a compound is found to easily penetrate the blood brain barrier, then it may be an effective neuro-active drug. But if that compound is not intended to be neuro-active, it may possess undesirable side effects such as drowsiness. Thus, the medicinal chemist should determine early in the drug development process whether or not a compound penetrates the blood brain barrier.

Typically, the ADMET/PK properties (absorption, distribution, metabolism, excretion, toxicity/pharmacokinetic properties) of a given compound are difficult to predict. As a result, one must resort to expensive and time-consuming in vitro and in vivo experiments to provide the necessary information. Sometimes these experiments are rather unreliable, particularly in the case of relatively insoluble compounds. In the current state of drug development, many drug candidates travel rather far toward commercialization before an intrinsic ADMET/PK flaw is discovered. The farther such compounds travel down the development pathway, the more wasted expense they represent.

This problem is not new. To address it, medicinal chemists have traditionally employed various logical or algorithmic tools. Probably, the best known of the simple logical tools is Lipinski's Rule of 5. C. A. Lipinski developed this rule of thumb for identifying drug-like compounds. The rule states that most drug-like compounds have a molecular weight of less than 500, cLogP of less than 5, a hydrogen bond donor count of less than 5, and a hydrogen bond acceptor count of less than 10. While very simple to use and surprisingly accurate, Lipinski's rule is still only a rough approximation. Further, it does not predict the manner in which a particular drug may be deficient if it falls outside the “sweet spot.” For example, it cannot predict whether a problem with a drug may be due to metabolism, absorption, solubility, or some other property. Thus, the medicinal chemist does not know whether to direct her redesign efforts toward correcting a metabolism problem, correcting an absorption problem, or correcting some other problem. Further, knowledge of whether a compound will or will not cross the blood brain barrier is important in determining whether the drug will have significant side effects.

At the other end of the scale from simple rules of thumb, are very complex, computationally intensive, models for predicting specific ADMET/PK properties. For example, Crevori et al. have developed a model of blood brain barrier penetration that analyzes the surface of a three-dimensional representation of a molecule to determine the energetics of its interaction with various molecular probes, such as water. See P. Crevori et al. “Predicting BBB Permeation from Three Dimensional Molecular Structure” J. Med. Chem. 43:2204-2216 (2000). In the end, even these very complex, computationally intensive models have only limited accuracy. It is estimated that the model of Crevori correctly predicts blood brain barrier penetration in about 90% of the cases.

In some cases, relatively simple and accurate models have been developed for single properties such as solubility. One rather simple and useful model for discriminating between soluble and insoluble drug candidates was developed at Glaxo Wellcome, Ltd. This model employed Linear Discriminant Analysis to provide a line in two-dimensional descriptor space separating relatively soluble compounds from relatively insoluble compounds. The two descriptors employed in the model were a molecular size-based descriptor (CMR or calculated molar refractivity) and a partitioning descriptor (log D, a pH dependent partition coefficient). A similarly simple two-dimensional descriptor model for predicting whether a compound would or would not be absorbed in the intestine was developed at Glaxo Wellcome. While these two-dimensional descriptor based models were intuitive and simple to use, they provided limited information about other medicine-relevant properties. Most notably, these models could not predict whether a compound would or would not penetrate the blood brain barrier.

What is needed therefore are additional simple and easy to use models for allowing the medicinal chemists to quickly discriminate between those compounds that have a relevant property of interest and those that do not.

### SUMMARY OF THE INVENTION

This invention provides descriptor-based models for predicting various activities of chemical compounds. Preferably, the models of this invention employ two or more chemical descriptors to characterize chemical compounds. From the descriptor values, the models predict certain activities. In many embodiments, those activities are biology-based activities such as ADMET/PK properties. An important activity that may be predicted is the ability of the compound to cross the blood brain barrier.

In a specific embodiment, the model predicts at least one of the following activities: a compound's solubility, its ability to be absorbed in the intestine, and its ability to cross the blood brain barrier. The descriptors of interest are typically, though not necessarily, physicochemical properties of the whole molecule. Examples include a log P or log D, molecular weight or related size-based descriptor, number of hydrogen bond donors and/or hydrogen bond acceptors, formal charge, lipophilicity, and the like.

Sometimes models of this invention are depicted as multi-dimensional graphs of descriptor space divided into two or more activity regions. From such graphs, a medicinal chemist or other researcher can quickly visualize descriptor modifications that will allow redesign of a particular drug to provide a desired combination of activities.

In one example, the model is presented as a graph of log P (descriptor 1) versus the sum of the hydrogen bond donors and acceptors (descriptor 2). A line on the graph divides the two dimensional descriptor space into a first region containing compounds that easily cross the blood brain barrier and a second region containing compounds that do not so easily cross the blood brain barrier. The graph may provide additional information, such as information about other activities. For example, another line on the graph may divide the two-dimensional space into a third region containing compounds that are relatively insoluble (in aqueous biological fluids) and a fourth region in which the compounds are relatively soluble. A third line on the graph may divide the descriptor space into a fifth region containing compounds that are relatively easily absorbed in the human intestine and a sixth region containing compounds that are not so easily absorbed by the intestine.

In a specific embodiment, the line associated with blood brain barrier penetration is approximately perpendicular to the axis specifying the sum of hydrogen bond donor and acceptor sites. This line intersects the hydrogen bond donor-acceptor axis at about a value of six. This equates to an amazingly simple rule for blood brain barrier penetration: the sum of the hydrogen bond acceptors and donors should be less than six.

One aspect of the invention pertains to a method of predicting whether an organic compound will penetrate the blood brain barrier significantly. The method may be characterized by as follows: (a) determining whether the compound has fewer than six hydrogen bond donors and hydrogen bond acceptors; and (b) based on whether the compound has fewer than six hydrogen bond donors and hydrogen bond acceptors, predicting whether the compound will penetrate the blood brain barrier significantly. The compound is predicted to penetrate the blood brain barrier significantly only if it has fewer than six hydrogen bond donors and hydrogen bond acceptors.

In some embodiments, this method is implemented on a computing device. This allows for high throughput analysis. Thus, the method may involve automatically repeating (a) and (b) for multiple different compounds. A computer implemented method may also employ the following: (i) receiving a representation of the compound, and (ii) analyzing the compound in a manner that automatically identifies hydrogen bond donors and hydrogen bond acceptors.

The computer implemented method may also redesign the compound by adding one or more hydrogen bond donors and/or hydrogen bond acceptors if the compound originally had fewer than six total hydrogen bond donors and acceptors and now has six or more total hydrogen bond donors and acceptors. Alternatively, the method may redesign the compound by removing one or more hydrogen bond donors and acceptors if the compound originally had six or more total hydrogen bond donors and hydrogen bond acceptors and now has fewer than six total hydrogen bond donors and acceptors.

A different aspect of the invention provides a method of analyzing a compound, employing the following operations: (a) determining a total number of hydrogen bond donors and hydrogen bond acceptors on the compound; (b) determining a partitioning property of the compound; (c) based on at least the total number of hydrogen bond donors and hydrogen bond acceptors, classifying the compound based upon its ability to penetrate the blood brain barrier. And based upon the total number of hydrogen bond donors and hydrogen bond acceptors and the partitioning property, classifying the compound according to either (i) its solubility, (ii) its ability to be absorbed in the intestine, or (iii) both.

In one embodiment, the method employs a model including a two-dimensional space of hydrogen bond donor and acceptor count versus partitioning property, and a first line through said two-dimensional space, which first line separates a first region containing compounds that substantially penetrate the blood brain barrier from a second region containing compounds that do not substantially penetrate the blood brain barrier. The first line is substantially perpendicular to an axis specifying the total number of hydrogen bond donors and hydrogen bond acceptors in the two-dimensional space and crosses the axis at a value of about six total hydrogen bond donors and hydrogen bond acceptors.

Yet another aspect of the invention pertains to computer program products including machine-readable media on which are provided program instructions for implementing the methods described above, in whole or in part. Any of the methods of this invention may be represented, in whole or in part, as program instructions that can be provided on such machine-readable media. In addition, the invention pertains to various combinations and arrangements of data generated and/or used as described herein. For example, data representing the multidimensional graphs of this invention is itself part of the invention when stored or conveyed on a machine-readable medium. The invention also pertains to apparatus that may be designed or configured to use the models of this invention and/or methods related to those described above.

These and other features of the present invention will be described in more detail below in the Detailed Description of the Invention and in conjunction with the following figures.

### BRIEF DESCRIPTION OF THE DRAWINGS

FIG. 1 is a two-dimensional graph of a model, in accordance with an embodiment of this invention, including regions of descriptor space identifying solubility, blood brain barrier penetration, and human intestinal absorption.

FIG. 2A is a flow chart depicting a process for predicting blood brain barrier penetration.

FIG. 2B is a flow chart for using a model of the type depicted in FIG. 1.

FIGS. 3A and 3B illustrate a computer system suitable for implementing embodiments of the present invention.

FIG. 4 is a block diagram of an Internet based system for “rescuing” therapeutic compounds in accordance with an embodiment of the present invention.

### DETAILED DESCRIPTION OF THE INVENTION

### OVERVIEW

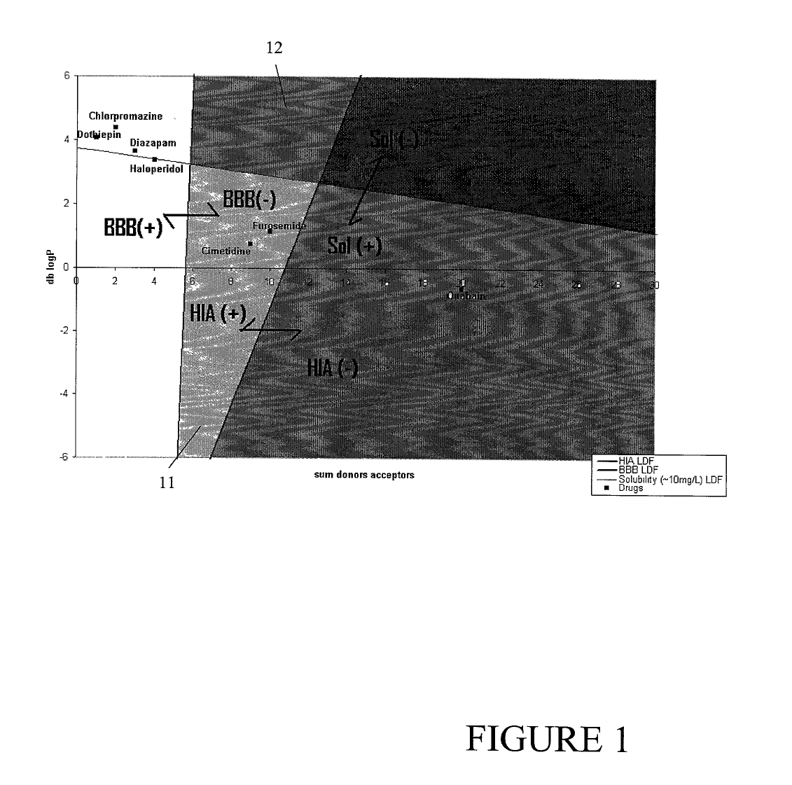

FIG. 1 depicts a two-dimensional model as an example of the type provided by this invention. As shown in the figure, a graph of the model includes two axes, a vertical axis representing variations in log P (the partition coefficient) and a horizontal axis representing variations in the sum of hydrogen bond donors and hydrogen bond acceptors. Points on the graph represent combinations of these two variables. More specifically, points on the graph represent individual compounds possessing the unique combinations of properties. Various therapeutic compounds are depicted as points on the graphs of FIG. 1. These are Chlorpromazine, Dothiepin, Diazepam, Haloperidol, Furosemide, Cimetidine, and Ouabain.

The two-dimensional space represented by the graph of FIG. 1 is divided into various regions defined by three separate lines through the two-dimensional space. The line with the most horizontal component defines a boundary between those compounds having a relatively low solubility (upper region) in those compounds having a relatively high solubility (lower region). In this example, compounds having a solubility in water of less than 25 micromolar, (about 10 milligrams per liter) are deemed to have low solubility. Compounds having solubility above this value are deemed to be soluble.

The vertical-most separation line in the two-dimensional space of FIG. 1 separates regions of high blood brain barrier penetration and low blood brain barrier penetration. To the right of this line reside compounds deemed to be unable to significantly penetrate the blood brain barrier. To the left reside compounds deemed to penetrate the blood brain barrier. Interestingly, the line is nearly vertical and passes through a point on the horizontal axis very close to a value of six total hydrogen bond donors and acceptors. Because this boundary line is nearly perpendicular to the hydrogen bond site axis, it results in an amazingly simple rule for blood brain barrier penetration: the sum of the hydrogen bond acceptors and donors should be less than six. It has been found that this simple rule alone, allows 89% correct classification of no penetration, and 82% correct classification of brain penetration.

The final dividing line in the graph of FIG. 1 is a primarily vertical line having a positive slope. This line divides the space into a region having good human intestinal absorption (HIA) characteristics (left region) and a region having relatively poor human intestinal absorption characteristics (right region). Depending upon which side of the line a compound falls, it may or may not be administered orally.

As can be seen from FIG. 1, the compounds Chlorpromazine, Dothiepin, Diazepam and Haloperidol have all been predicted to penetrate the blood brain barrier and be relatively insoluble (while also being absorbed well by the human intestine). The compounds Cimetidine and Furosemide have been correctly predicted to be well absorbed in the intestine but not penetrate the blood brain barrier. Also, these compounds are relatively soluble. Finally, the compound Ouabain has been correctly predicted to be soluble but not well absorbed in the intestine and also not penetrate the blood brain barrier.

Because the molecular descriptors used in this model are very easy to interpret and because the model enables easy visualization of the required descriptor space, a chemist can easily identify potential problems with a therapeutic candidate and suggest new structures meeting a desired profile. For example, if the profile of a new chemical entity requires oral administration but no central nervous system penetration (to minimize unwelcome side effects) then the medicinal chemist will aim for compounds in the area of chemical space indicated by the reference number 11 of FIG. 1. If a new chemical entity should fall within the region 12, the chemist will realize that the compound will have a relatively low aqueous solubility (less than about 10 milligrams per liter). The chemist can then try to redesign the compound to fall in region 11 (to reduce logP for example) or she can keep the compound but try to develop an appropriate formulation for administration (to account for the low solubility).

The information contained in models of this invention (such as the one depicted in FIG. 1) can be used in various formats. In certain preferred embodiments, the information provided in a model is stored or transmitted as data or a data structure that can be accessed by a computing device. In the case of FIG. 1, the data specifies the relevant range of two-dimensional space defined by a partitioning property and a count of hydrogen bond donors and acceptors. The data also specifies how the relevant two-dimensional space is divided into regions relevant to the medicinal chemist (e.g., regions specifying blood brain barrier penetration, human intestinal absorption, and solubility). Thus, one aspect of the invention pertains to such data or data structures when stored or otherwise provided on a machine-readable medium. Note that the invention is not so limited, and may be used merely as a visual depiction on paper or other medium employed by a medicinal chemist.

The model depicted in FIG. 1 was generated using multiple training sets of compounds for which the partition coefficient was known and for which one or more of the activities have been characterized: blood brain barrier penetration, human intestinal absorption, and solubility. A linear discriminate analysis was employed for this purpose. In accordance with this invention, a similar process may be employed to generate models employing slightly different combinations of descriptors and activities. Note that the invention is not limited to linear discriminate analysis as a technique for determining the locations of boundary lines between regions of activity. Nor is the invention limited to “binary” models in which chemical entities are classified on a yes/no basis. For example, some models of this invention may employ multiple solubility ranges. The models may also employ multiple HIA ranges, for example.

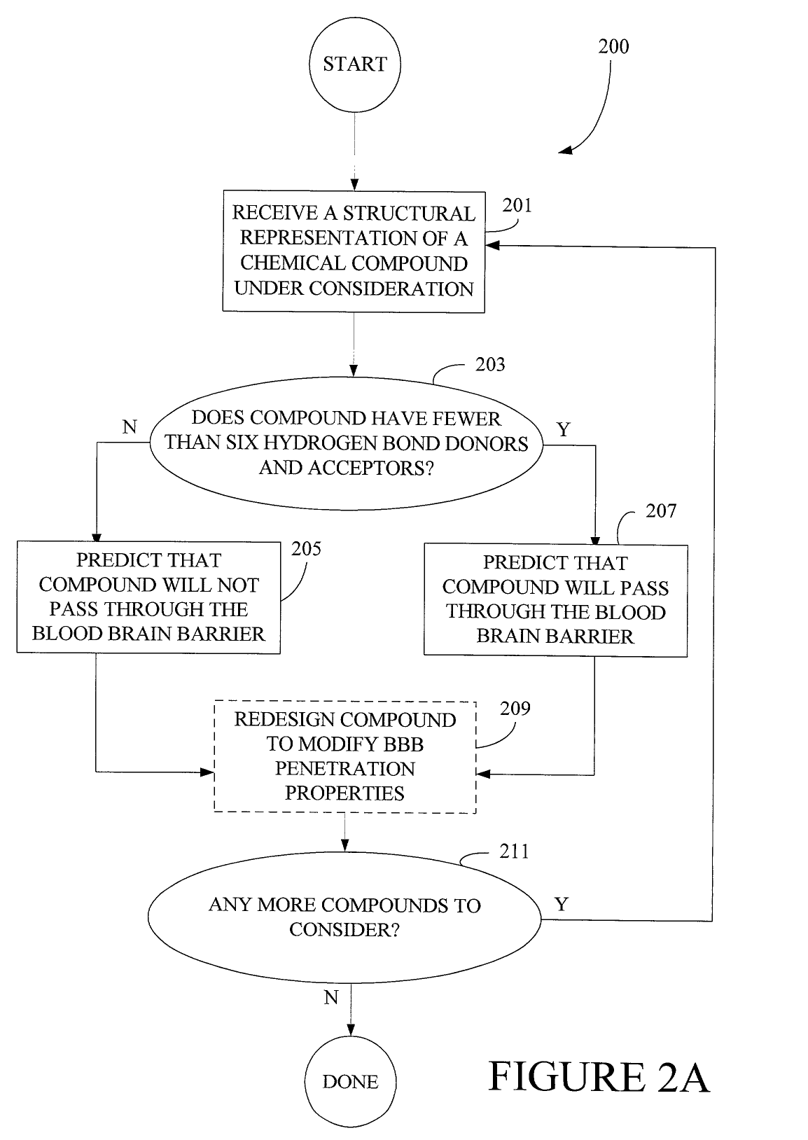

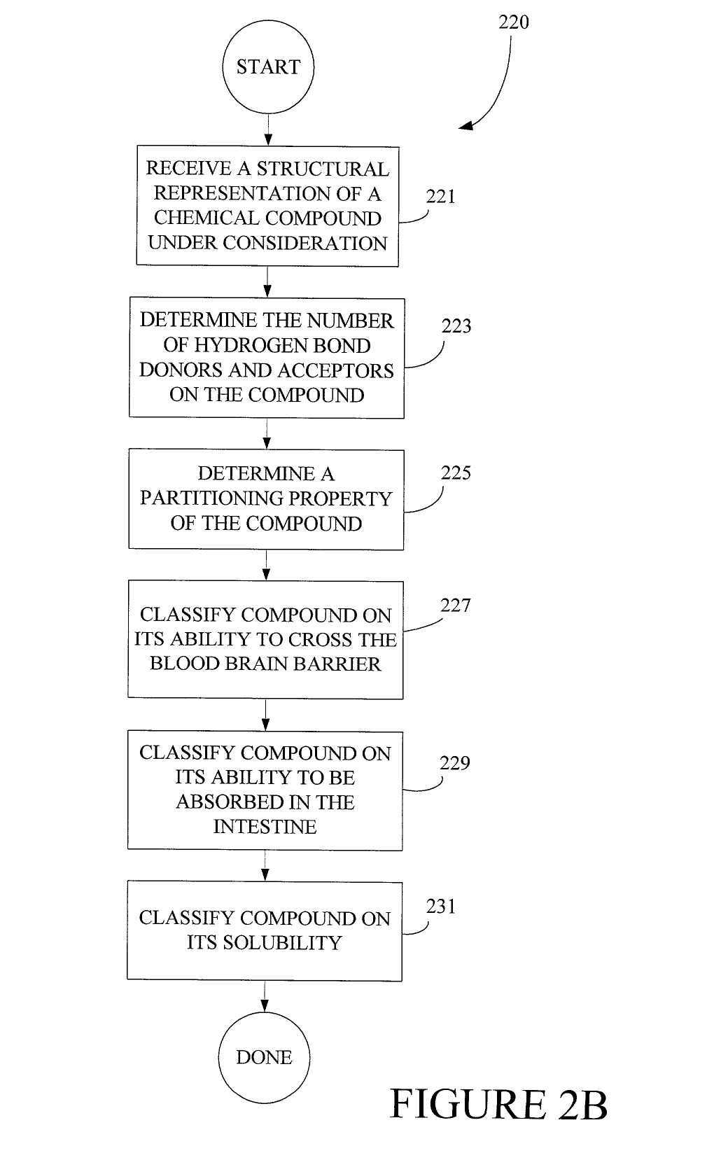

FIG. 1 and the associated discussion above lead to certain general applications for this invention. Two of these applications are depicted in FIGS. 2A and 2B.

Referring first to FIG. 2A, a process for depicting blood brain barrier penetration is identified by reference number 200. As shown there, the process begins at 201 by receiving a structural representation of a chemical compound under consideration. From this representation, a computing system or an individual can determine whether the compound has fewer than six hydrogen bond donors and acceptors. See decision 203. (In an alternative embodiment, the hydrogen bond donor/acceptor count is provided directly to a computer system, without first providing the structural representation.)

If the compound is found to have fewer than six hydrogen bond donors and acceptors, it is predicted that the compound will, in fact, pass through the blood brain barrier. See 207. If, on the other hand, the compound is found to have six or more hydrogen bond donors and acceptors, it will be predicted the compound will not actually pass through the blood brain barrier. See 205.

Based upon the prediction made at 205 or 207, it may be desirable to redesign the compound by modifying its chemical structures slightly. See 209. The modification is intended to tailor the compound's blood brain barrier penetration properties to the needs of the research effort. This is an optional procedure, depending upon the goals of the research effort and the predicted properties of the compound under consideration. Assuming that a compound is to be redesigned, the structural modification is chosen to change the sum total count of hydrogen bond donors and acceptors on the molecule under consideration. Depending on the desired properties, the structural modification may increase the donor/acceptor count to six or more, or it may decrease the count to less than six. Either way, this should be done in a manner designed to preserve the therapeutic effectiveness of the compound. In one example, this is accomplished by maintaining a pharmacophore representing the relevant binding structure or other therapeutic feature of the molecule. Hydrogen bond donors/acceptors may be added or subtracted, so long as the changes do not significantly impact the desired pharmacophore.

Note that the above analysis may be performed as a high throughput screening operation for rapidly assessing the potential value of (and redesigning as necessary) great numbers of chemical compounds. To this end, the process 200 may consider numerous compounds, one after the other. As depicted in FIG. 2A, process 200 may be performed multiple times, once for each compound in question. A control operation 211 allows for this looping. When no further compounds are to be considered, decision 211 is answered in the negative, and the process is complete.

FIG. 2B presents another process (process 220) for using a model of the type depicted in FIG. 1. As shown there, the process begins at 221 by receiving a structural representation of a chemical compound under consideration. This representation may be as simple or as detailed as necessary. With a model employing the descriptors shown in FIG. 1, the structural representation need only provide enough information to discern the total count of hydrogen bond donors and acceptors (and possibly the partition coefficient). As with FIG. 2A, an alternative embodiment allows for receipt of the relevant descriptor information directly, thus avoiding the need to extract the information from a chemical structure.

Assuming that information is obtained indirectly via a structure, an operation 223 determines the total number of hydrogen bond donors and acceptors on the compound. This may be accomplished in numerous ways. Generally, hydrogen bond donors include hydrogen atoms on hydroxyl groups, primary and secondary amines, mercaptan groups, etc. Hydrogen bond acceptors include oxygen, nitrogen, sulfur, etc. on such groups. Whenever one of these groups is found on a molecule, the donor/acceptor count is incremented by one.

The process also determines a partitioning property of the compound. See 225. As described below, this partitioning property may take various forms such as logP and logD. Basically, these properties define the ability of a compound to partition between an aqueous phase and a non-aqueous phase.

Next, at 227, the process classifies the compound under consideration based on its ability to cross the blood brain barrier. With reference to FIG. 1, this may simply involve determining on which side of the vertical blood brain barrier boundary line a compound lies.

In addition, at 229, the process classifies the compound based on its ability to be absorbed in the intestine. Again, with reference to FIG. 1, this involves determining on which side of the HIA line a compound lies.

Finally, the process 220 classifies the compound of interest based on its aqueous solubility. See 231. Using the model depicted in FIG. 1, this simply involves determining which side of the solubility line the compound lies on.

Definitions

At this point, to assist in understanding the concepts presented herein, the following simple explanations are provided for some terms. The scope of the invention should not necessarily be limited by the following examples.

“Physicochemical property” (or sometimes just “property”) refers to a particular physical and/or chemical property of a compound under consideration. The property may pertain to the compound as a whole, a region or fragment of the compound, or individual atoms within the compound. Examples of whole compound physicochemical properties include partition coefficient (P and logP), pH dependent partition coefficient (D and logD), size based properties such as molecular weight (MW) and calculated molar refractivity (CMR), formal charge (FC), number of hydrogen bond donors and/or number of hydrogen bond acceptors, the presence or absence of particular chemical motifs and moieties such as aromatic centers, and the like. Examples of atom specific physicochemical properties include chemical information about a site atom, neighbor atoms, partial charge, total charge, bond length, and the like.

“Descriptor” refers to a variable or value representing a property of a particular compound. Thus, the term is closely related to, and in a sense overlaps with, “physicochemical property.” Descriptors may be viewed as quantitative or textual representations of properties. They appear in expressions or models for predicting “activities” of a particular compound. A potentially infinite number of descriptors may characterize a compound. Multivariate models employ two or more descriptors to predict the activity of a compound.

“Activity” refers to an important characteristic of a compound. In a sense, an activity is like a “property” of a compound. However, in the context of this invention, activity usually refers to a biochemical, biological, and/or therapeutic behavior of a compound. Also, the activity of a compound is usually a characteristic that is to be predicted. Often, an activity serves as a dependent variable related to descriptors, which are independent variables. The models of this invention predict activity from descriptor values. Examples of activities that may be predicted with the models of this invention include solubility, penetration of the blood brain barrier, intestinal absorption, and any other particular ADMET/PK characteristic.

Depending on how a model is constructed, activity may take the form of a specific numerical value (e.g., percent absorption or penetration) or a threshold or filter (e.g., penetrates or does not penetrate; is soluble or is not soluble). In some embodiments, a model of this invention will divide an activity into more than two discrete regions. For example, if the activity is solubility, the model may bin compounds into solubilities (1) less than 0.1 mg/l, (2) 0.1-10 mg/l, (3) 10-100 mg/l, and (4) greater than 100 mg/l.

A “Model” is a mathematical or logical representation of a physical and/or chemical relationship. Models may predict an activity from one or more descriptors of physical and/or chemical properties. In other words, such models treat an activity as a dependent variable and descriptors as independent variables. Thus, the model is itself a mathematical or logical relationship.

Models can take many different forms. In one preferred embodiment, the model form is a two or three-dimensional graph having various regions of activity defined by lines or curves. These boundary lines may be obtained using Linear Discriminant Analysis (LDA) for example. Each separate region of the descriptor space includes compounds predicted to have a particular property like blood brain barrier penetration, human intestinal absorption, solubility in aqueous media, etc.

Models are typically developed from a training set of chemical compounds or other entities that provide a good representation of the underlying physical/chemical relationship to be modeled. The activities and descriptors form members of the training set and are used to develop the mathematical/logical relationship between activity and descriptors. This relationship is typically validated prior to use for predicting activity of new compounds.

Descriptors (Independent Variables)

This invention employs models of chemical activity space employing descriptor values as a frame of reference. In other words, descriptor values serve as axes for the chemical space. Various descriptors may be chosen for the purpose. As indicated in the discussion of FIG. 1 above, the descriptor axes may be partition coefficient (logP) values and sum hydrogen bond donors/acceptor values. These two descriptors have been found to do a very good job of separating compounds based on blood brain barrier penetration, human intestinal absorption, and aqueous solubility. Variations on these descriptors such as the use of log D as an axis may also give good separations. For models of other activities, other descriptor combinations will be appropriate.

A hydrogen bond donor is generally defined as a hydrogen atom covalently bound to an electronegative atom or group such as an oxygen atom, a sulfur atom, or a nitrogen atom. Examples of hydrogen bond donors include the hydrogen atoms in hydroxyl groups, carboxylate groups, amides, amines, mercaptans, and the like. A hydrogen bond acceptor on a compound is generally defined as an electronegative atom having a free electron pair. Often oxygen, nitrogen, and sulfur heteroatoms serve as hydrogen bond acceptors. Examples include either oxygen atoms, disulfide sulfur atoms, and amine nitrogen atoms (in primary, secondary, or tertiary amines). Lipinski's rule of five suggests that most drug-like compounds have less than ten hydrogen bond donors and/or hydrogen bond acceptors.

A skilled chemist can quickly identify such donors and acceptors on an arbitrary chemical compound. Similarly, numerous computation tools can make this assessment. Examples include Cerius 2 (Accelerys Inc), MOE (Chemical Computing Group Inc).

A partition coefficient generally represents the degree to which a compound separates between two immiscible phases: one aqueous and the other non-aqueous. Various types of partition coefficients are defined. The variations arise for the most part in the choice of non-aqueous phase and in the amount of buffering employed. In a typical approach, the non-aqueous phase is n-octanol and the aqueous phase is a phosphate buffer of 7.4 pH.

Numerically, a partition coefficient is defined as the ratio of the solute in the organic phase to solute in the aqueous phase. Partition coefficients can be easily measured, but must be done in a consistent manner. Some models can predict/calculate the partition coefficients of arbitrary compounds. Typically, the calculated and measured partition coefficients are provided as the logarithm of the partition coefficient, or logP. In the example of FIG. 1, the partition coefficient is a calculated log P, obtained using molecular descriptors.

A variation of the partition coefficient is the pH dependent partition coefficient (distribution coefficient, D), typically presented as logD. This is typically measured experimentally in a similar manner to log P except that the pH of the aqueous buffer is altered, and measurement taken over a pH range (1-14). Alternatively, a calculated log D may be obtained, but this requires additional knowledge or calculation of the dissociation constants associated with all the ionizable groups within a given molecule. Due to this, calculation of log D is often less reliable than that of log P.

Another useful class of descriptors is the size-based descriptors. These include molecular weight, calculated molar refractivity (CMR) and McGowans volume (Vx) to name just three. These descriptors are highly inter-correlated and essentially relate to the size of the molecule. Molar Refractivity can be considered as the sum of either atom or bond refractivities. This sum, which can be obtained directly from the compound's structure, should then equal the value given by the Lorenz-Lorenz equation when the measured values of density and refractive index have been inserted.

$$M R = \frac{{μ}^{2} - 1}{{μ}^{2} + 2} \cdot \frac{M}{d}$$

Other descriptors that may be used in a similar way in other LDA models of this type are those physico-chemical descriptors that also have strong correlation with size and lipophilicity. For example certain combinations of Polar Surface Area (PSA), hydrophobic volume, hydrophilic volume, the sum of oxygen and nitrogen atoms, log P, log D and other size related descriptors may in some applications be used in place of the sum of hydrogen bond donors and acceptors and log P used as described above. For many applications, the two descriptors chosen give the best overall modeling statistics.

Activities (Dependent Variables)

Generally, an activity that is predicted by a model of this invention is a biological or chemical property. Such activity is often not easy to measure or predict without the benefit of such models. Activities of particular interest include permeation rate limited activities. These activities represent a compartmentalization of a compound within tissues or organs. Typically, the two sides of a tissue or organ boundary will vary in fat content or types of binding proteins. As indicated, specific examples of activities that can be predicted with the aid of descriptor based models of this invention include solubility in an aqueous medium, blood brain barrier penetration, intestinal absorption, and the like.

Regarding solubility, the models of this invention can predict/classify a compound as either high or low solubility based on an arbitrary cutoff point such as 10 milligrams per liter. Alternatively, the models can classify a compound within a given range of solubility. For example, it has been found that models like those described herein can accurately segregate compounds into the four following solubility classes: less than 0.1 milligram per liter, 0.1-1 milligram per liter, 1-10 milligrams per liter, 10-100 milligrams per liter, and greater than 100 milligrams per liter.

To develop a model that can accurately predict solubility, a training set of compounds is required for which accurate solubility measures have been developed. Solubility can be measured in many different ways. Most basically, solubility is measured at a given temperature by adding enough solute to a given solvent so that some solid remains in the solvent. The concentration of solute in this saturated solution is determined by an appropriate analytical means.

With regard to intestinal absorption and subsequent distribution, the compound in question must cross the epithelial cells that line the gastrointestinal tract, and then it must cross the endothelial cells that line the blood capillaries that perfuse the target organs. Such traversing can occur via the paracellular (in between cells) or the transcellular (through cells) pathway. Since only small hydrophilic molecules (MW less than 350) can use the paracellular route, the majority of compounds cross cell membrane barriers via transcellular routes. Transcellular routes include passive transport and active transport. At the intestinal epithelium, compounds are absorbed primarily via passive diffusion across the vast surface area of the densely packed microvilli at the apical brush border membrane, and via transport mediated by selective transporter proteins.

Many drugs and drug candidates will fall into a higher MW category (e.g., MW greater than 300), so that appropriate LDA models need not always account for paracellular mechanisms. Of course, the training set may include compounds over a wide range of molecular weights that work by both mechanisms. So the resulting model (such as the one shown in FIG. 1) can cover HIA by transcellular and paracellular routes.

Further, the models of this invention may be designed to generally handle both passive and active transport (whether across the intestinal line or some other boundary such as the blood brain barrier). In such cases, the training set will not be limited to compounds that predominately pass by either passive or active transport. In other embodiments of this invention, the training set will be limited to compounds known to have a predominant transport mechanism of either passive or active transport, but not both. In more specific embodiments, the training set will be limited to compounds known to employ a particular transporter protein during active transport.

Intestinal absorption can be measured by various techniques. Some involve administering compounds to animals and determining how much of the administered compound passes through the intestine. A currently popular in vitro technique employs caco-2 cells (immortal human intestinal epithelial cells) on multi-well plates. The cells form a consistent bilayer in the wells. Different compounds under test are administered to each well of the plate. After a time, the amount of compound that has passed through the intestinal cells is measured.

The units of intestinal absorption are normally percent absorption in the intestine. In other words, the amount of compound absorbed per 100 units administered to a patient constitutes the compound's absorption. The transition point between compounds that are considered to be absorbed and those that are not can range from about 5% to about 50% in normal analyses. The models of this invention may also make use of thresholds in this range. For a given purpose, a researcher may choose a particular threshold. For a different purpose, a different researcher may choose a different threshold. In a specific embodiment herein, the dividing line between good and bad absorption is drawn at about 30% absorption. This is the value employed to generate the model depicted in FIG. 1.

Other activities modeled in accordance with this invention include distribution and/or excretion of particular compounds. “Distribution” includes Blood-Brain-Barrier (BBB) penetration, protein binding in the blood, receptor binding and drug transport across cell membranes. The active and passive transport models are similar to those discussed above with respect to absorption models. Modeling of excretion includes hepatic and renal excretion, which are also mediated by active and passive transport mechanisms, and require similar models. For instance, P-gp is expressed on the brush border and biliary face of proximal tubule cells in the kidney and hepatocytes, respectively, consistent with a role for this active transporter in the excretion of compounds into the urine and bile.

In general, distribution and excretion can be modeled as described above, preferably using a collection of suitable descriptors. The descriptors identify key properties and/or structural motifs that strongly impact a distribution and/or excretion mechanism.

For this invention, blood brain barrier penetration is a particularly important form of distribution. This is because the parameter is difficult to measure and is critically important to drug development. If a research organization intends a new chemical entity to act via neurological pathway, then the chemical must effectively penetrate the blood brain barrier. If on the other hand, the organization wishes to develop an allergy medication or other therapeutic that does not act on the central nervous system, then the chemical compound should not pass the blood brain barrier. Otherwise, unwanted side effects may result. For example, many antihistamines have the unwanted side effect of drowsiness because they penetrate the central nervous system and interact with the brain.

In predicting whether a compound will pass through the blood brain barrier, some threshold measure or degree of passage should be used. Unfortunately, measuring penetration of the blood brain barrier is not an easy task, susceptible of rigorous quantitative analysis. However, most compounds lie clearly on one side or the other in ability to penetrate the blood brain barrier. Thus, the actual quantitative threshold between a compound that crosses and one that does not may be somewhat arbitrary. Generally speaking, psychoactive therapeutics are deemed to cross the blood brain barrier. So training sets may include binary information (passes/does not pass) based on whether the compound under consideration is known to have a psychoactive effect.

Predicting blood brain barrier permeation remains a challenge in drug design, since it is impossible to determine experimentally for large numbers of potential pre-clinical compounds. The study by Cruciani et al, was conducted to demonstrate the value of descriptors derived from 3D molecular fields. The method used (VolSurf) transforms the calculated 3D fields into 72 descriptors and correlates them to experimental permeation by a discriminant partial least squares procedure. It is claimed that this model predicts more than 90% of the BBB permeation data correctly. This method is time consuming and complex.

While most effort in developing therapeutic compounds is directed at humans, that is not always the case. So while most examples presented herein pertain to humans, the invention is not so limited. It generally applies to models for any biological entity—although it is particularly valuable for predicting blood brain barrier penetration. So in most cases, the models will be pertinent to animal species having a central nervous system. Because of inter-species differences, the training sets used to develop the models should specify biological activities for the species of interest. For example, a therapeutic drug being developed for a neurological condition in cattle should employ a model developed from a training set having blood brain barrier penetration data for bovines.

Generating the Models

Materials for which models of this invention may predict a pertinent activity include most any compound introduced (such as by ingestion or inhalation) into a living organism. Particularly preferred compounds for analysis are potential therapeutics considered in a drug discovery effort. In developing a model of activity for such compounds, one should carefully choose a training set. A large group of structurally diverse chemical compounds should be used. Generally, a training set member may be any compound that has been synthesized and has had its pertinent activities characterized.

The specific compounds chosen for the training set may also be focused on the chemical structural space relevant to the model. For example, if a model is to be developed for potential therapeutic compounds taken orally, then the training set should include various small organic drug-like molecules.

The training set size depends in part on the amount of diversity among the members of the group. Structural “diversity” means that the compounds of the set have a wide range of different functional groups and functional group environments. Such diversity may be obtained using a wide range of “scaffolds” (e.g., various ring systems) and “building blocks” (e.g., substitutions). Since this invention pertains to models that predict activities based on certain descriptors, the members of the training set should exhibit a wide range of values for such descriptors—regardless of other measures of diversity.

In some cases, distinct training sets are used for developing separate types of models. As indicated above, model types include blood brain barrier models, bioavailability models, etc. The training set for a blood brain barrier penetration model should include compounds that penetrate as well as those that do not. Training sets having only members that cross the blood brain barrier would typically fail to produce a useful model.

In the model depicted in FIG. 1, 104 compounds having known blood brain barrier penetrations capabilities (see P. Crevori, et al., J. Med. Chem. 43:2204-2216 (2000)) were employed as the training set. For each of these compounds Crevori et al. specified a degree of blood brain barrier penetration in terms of three-dimensional molecular descriptors based on the energetics of various positions on the three-dimensional molecular surface with respect to water. For developing the model of FIG. 1, the same data and classifications were used as those given in table 2 (repeats removed) in the paper cited above by Crevori et al. The model so generated was validated with a set of known CNS-active compounds. Some of those compounds are shown in FIG. 1.

For developing the solubility aspect of the model, approximately 2500 compounds having known solubilities were used as the training set. These compounds and their associated solubility values were obtained from commercially available databases, such as that supplied by the Syracuse Research Corporation (SRC) PhysProp database. Approximately 10 milligrams per liter was chosen as the solubility cutoff point between “soluble” and “insoluble” compounds for purposes of the model depicted in FIG. 1.

Finally, for the HIA component of the FIG. 1 model, approximately 240 compounds with HIA values obtained from Abraham et al, (J Pharm Sci, Vol 90, No 6, pp 749-784) were used in the training set. The cutoff between absorption and no absorption was chosen to be 30 percent absorption.

When the appropriate training set has been selected and characterized by activity and pertinent descriptors, then the model can be generated by an appropriate data fitting technique. Associating activity with particular descriptors generates the model. Generally, “association” represents an attempt to find a relationship between the two groups of variables. One set of variables is the dependent set of variables and these are a function of the other set, the independent set of variables. In this invention, the dependent variables are activities and the independent variables are the descriptor values.

Preferably, the model association is generated using Linear Discriminant Analysis, as mentioned above. LDA divides descriptor space into regions or activity separated by lines. An observation is classified into a group if the squared distance (also called the Mahalanobis distance) of observation to the group center (mean) is the minimum. An assumption is made that covariance matrices are equal for all groups. There is a unique part of the squared distance formula for each group and that is called the linear discriminant function for that group. For any observation, the group with the smallest squared distance has the largest linear discriminant function and the observation is then classified into this group. Linear discriminant analysis has the property of symmetric squared distance: the linear discriminant function of group i evaluated with the mean of group j is equal to the linear discriminant function of group j evaluated with the mean of group i. The software used to perform LDA (in the embodiment of FIG. 1) was supplied by Minitab Inc, 3081 Enterprise Drive, State College, Pa. 16801.

Examples of other data fitting techniques that may be used in alternative embodiments of this invention include various regression techniques, partial least squares, principal component analysis, back-propagation neural networks and genetic algorithms. Principal component analysis is described in P. Geladi, Anal. Chim. Acta, 185, 1, (1986) which is hereby incorporated by reference.

General Description of Models

Many models of this invention represent compounds based on two descriptors: one pertaining to a partitioning property and another pertaining to a count of hydrogen bond donors and/or hydrogen bond acceptors. Thus, it is convenient to represent all compounds in a two-dimensional space defined by one partitioning property axis and one hydrogen bond donor/acceptor axis, as shown in FIG. 1. While the specific values represented on the two axes can take many different forms, in one preferred embodiment, the partitioning property is logP and the hydrogen bond donor acceptor descriptor is a sum of all hydrogen bond donors and hydrogen bond acceptors on the compound. In alternative embodiments, the partitioning property may be logD or simply P or D. Further, in such alternative embodiments, the hydrogen bond donor/acceptor axis may represent some subset of the total hydrogen bond donors and hydrogen bond acceptors on the compounds. Particular chemical types of donors and acceptors may define the subset, for example.

As for the activities of interest, the two-dimensional space described above should include multiple regions of different activity (dependent variables). The activities include any one or more of blood brain barrier penetration, intestinal absorption, and solubility. Other suitable activities for modeling by this invention may include any biological process that involves permeation as its rate-limiting step.

The two-dimensional space of interest can be separated into multiple activity regions by lines or curves. Linear discriminate analysis will give lines, but other techniques may separate the region using curves or various discontinuous functions.

Understand that the two-dimensional descriptor space described above (partitioning property versus hydrogen bond sites) may be part of a three-dimensional or higher dimensional descriptor space. In a specific embodiment, the third dimension can be a size based parameter such as molecular weight or calculated molar refractivity, formal charge, the presence of a particular chemical motif, partial charge, etc. The fourth or higher dimension, if employed, could also be selected from this list.

The dependent variables (activities) represented in the model may be provided in binary form (e.g., penetrates or does not penetrate) as depicted in FIG. 1, or they may be represented as three or more discrete regions. One example of this second format employs four discrete solubility ranges, as described above.

Use of the Models

The model of this invention is particularly useful to medicinal chemists. Considering the model of FIG. 1 as an example, a medicinal chemist can use each of the three depicted activities to make decisions about the mode of administration and/or compound redesign.

Solubility data of a lead compound can specify a route of delivery. If the compound is highly insoluble and oral administration is desired, then the research team may opt for a syrup or gel cap form of administration. Alternatively, the research team may determine that an intravenous administration will work best.

Solubility data can also be used to identify those compounds that will be difficult to analyze and/or screen. Highly insoluble compounds often give false results or poor recoveries during normal testing. Thus, if a model of this invention predicts a compound will have a particularly low solubility, then the research team knows to treat in vitro data for that compound as suspect. More rigorous analysis will be merited if the compound looks particularly promising.

Intestinal absorption predictions provided by the models of this invention can be used to quickly decide whether oral administration will be a suitable route. If the model predicts the compound is unlikely to be absorbed in the intestine, then the research team may decide that intravenous administration or suppository administration should be employed. Appropriate formulations can then be developed.

Regarding blood brain barrier penetration, the activity predicted by the model can be used by a medicinal chemist to determine whether a potential CNS-active compound will actually be useful. If the compound will not likely cross the blood brain barrier, then it must either be redesigned in a manner that will allow it to cross the blood brain barrier or be abandoned. If a compound is not intended to be CNS-active but is predicted to cross the blood brain barrier, then the research team must consider the possibility of CNS side effects such as drowsiness. If such compound is predicted to cross the blood brain barrier, then its side affects must be rigorously scrutinized. Alternatively, the compound could be redesigned to not cross the blood brain barrier but preserve its therapeutic activity.

When the models of this invention are automated in a computer system such as that described below, high-throughput screening of numerous potential therapeutic compounds can be performed for appropriate ADMET/PK properties. Only those compounds that appear to have the proper combination of ADMET/PK properties (e.g., HIA and blood brain barrier penetration) are selected for further testing. Thus, the models of this invention may be employed to filter out dead end compounds before expending substantial effort and money on in vivo testing or, in some cases, even advanced in vitro testing. Generally, the models of this invention may be employed to screen potential therapeutic compounds at any stage in the drug design process, although most value will likely be had in the early and middle stages of drug development.

Another extremely important application of the present invention is in guided redesign of potential therapeutic compounds that have ADMET/PK difficulties. Using the models of this invention, a skilled chemist or moderately sophisticated software routine can propose modified chemical structures having improved ADMET/PK properties. The suggested modifications are made within the descriptor space used to represent activities in two or multi-dimensional space. For example, if a compound appears to have very promising interactions with a receptor in neurons but cannot penetrate the blood brain barrier, the models of this invention in conjunction with a redesign effort could reduce the number of hydrogen bond donors/acceptors on the compound to produce a new compound that can cross the blood brain barrier, and preserve the therapeutic effectiveness. Preferably, redesign software employed with this invention would act as an expert system allowing adjustments in the compound within the descriptor space of interest, while preserving the therapeutic effectiveness of a base compound (having inferior ADMET/PK properties). To preserve the therapeutic effectiveness, the systems of this invention may, for example, make use of a pharmacophore that is presumed responsible for the therapeutic activity. The redesign software will be constrained to make modifications to regions of the compound that are not associated with the pharmacophore of interest, thereby presumably preserving the therapeutic activity.

Hardware and Software

Certain embodiments of the present invention employ processes acting or acting under control of data stored in or transferred through one or more computer systems. Embodiments of the present invention also relate to an apparatus for performing these operations. This apparatus may be specially designed and/or constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein, or it may be more convenient to construct a more specialized apparatus to perform the required method steps. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, and magnetic tape; optical media such as CD-ROM devices and holographic devices; magneto-optical media; semiconductor memory devices, and hardware devices that are specially configured to store and perform program instructions, such as read-only memory devices (ROM) and random access memory (RAM), and sometimes application-specific integrated circuits (ASICs), programmable logic devices (PLDs) and signal transmission media for delivering computer-readable instructions, such as local area networks, wide area networks, and the Internet. The data and program instructions of this invention may also be embodied on a carrier wave or other transport medium (e.g., optical lines, electrical lines, and/or airwaves). Examples of program instructions include both machine code, such as produced by a compiler, and files containing higher level code that may be executed by the computer using an interpreter.

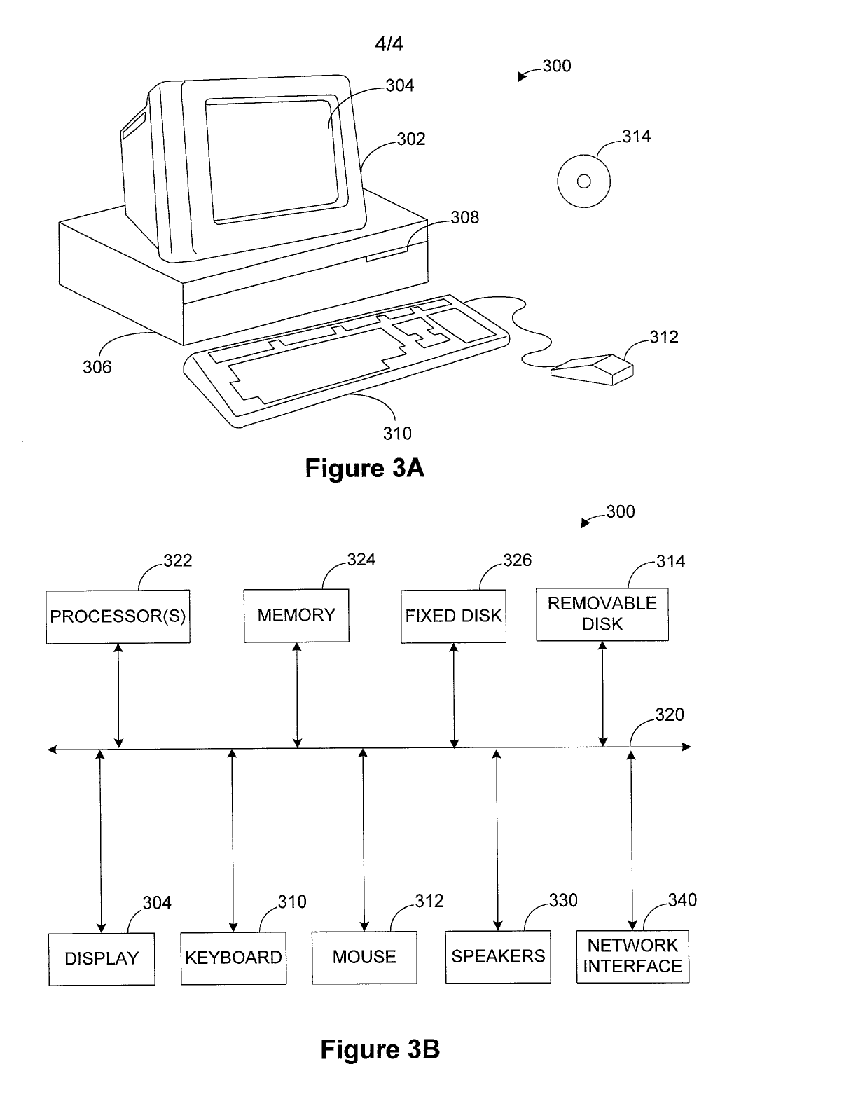

FIGS. 3A and 3B illustrate a computer system 300 suitable for implementing embodiments of the present invention. FIG. 3A shows one possible physical form of the computer system. Of course, the computer system may have many physical forms ranging from an integrated circuit, a printed circuit board and a small handheld device up to a very large super computer. Computer system 300 includes a monitor 302, a display 304, a housing 306, a disk drive 308, a keyboard 310 and a mouse 312. Disk 314 is one example of a computer-readable medium used to transfer data to and from computer system 300.

FIG. 3B is a block diagram of certain logical components of computer system 300. Attached to system bus 320 are a wide variety of subsystems. Processor(s) 322 (also referred to as central processing units, or CPUs) are coupled to storage devices including memory 324. Memory 324 includes random access memory (RAM) and read-only memory (ROM). ROM acts to transfer data and instructions uni-directionally to the CPU and RAM is used typically to transfer data and instructions in a bi-directional manner. Both of these types of memories may include any suitable computer-readable medium, including those described above. A fixed disk 326 is also coupled bi-directionally to CPU 322; it provides additional data storage capacity and may also include any of the computer-readable media described below. Fixed disk 326 may be used to store programs, data and the like and is typically a secondary storage medium (such as a hard disk) that is slower than primary storage. It will be appreciated that the information retained within fixed disk 326, may, in appropriate cases, be incorporated in standard fashion as virtual memory in memory 324. Removable disk 314 may take the form of any of the computer-readable media described below.

CPU 322 is also coupled to a variety of input/output devices such as display 304, keyboard 310, mouse 312 and speakers 330. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, or other computers. CPU 322 optionally may be coupled to another computer or telecommunications network using network interface 340. With such a network interface, it is contemplated that the CPU might receive information from the network, or might output information to the network in the course of performing the above-described method steps. Furthermore, method embodiments of the present invention may execute solely upon CPU 322 or may execute over a network such as the Internet in conjunction with a remote CPU that shares a portion of the processing.

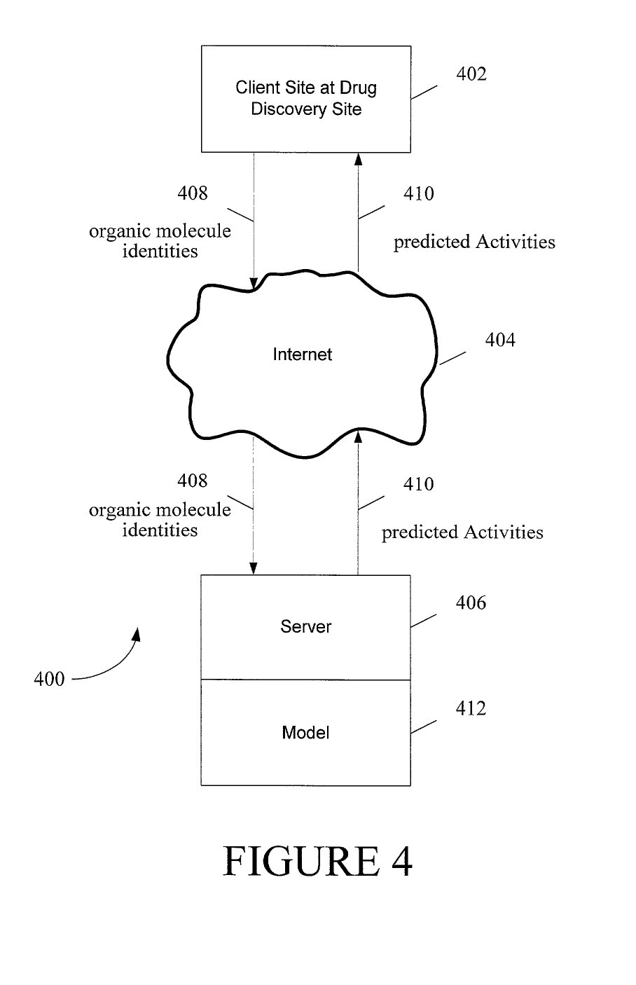

FIG. 4 is a schematic illustration of an Internet-based embodiment of the current invention. See 400. According to a specific embodiment, a client 402, at a drug discovery site, for example, sends data 408 identifying organic molecules 408 to a processing server, 406 via the Internet 404. The organic molecules are simply the molecules that the client wishes to have analyzed by the current invention. At the processing server 406, the molecules of interest are analyzed by a model 412, which determines whether the molecules are likely to pass the blood brain barrier and/or the intestinal wall. It may also predict the solubility of the compound or other physicochemical and/or biological activity. As indicated, the processing server may also redesign compounds to improve their ADMET/PK properties.

After the analysis, the predicted activities 410 (and any other appropriate information) are sent via the Internet 404 back to the client 402. The computer system illustrated in FIGS. 3A and 3B is suitable both for the client 402 and the processing server 406. In a specific embodiment, standard transmission protocols such as TCP/IP (transmission control protocol/internet protocol) are used to communicate between the client 402 and processing server 406. Security measures such as SSL (secure socket layer), VPN (virtual private network) and encryption methods (e.g., public key encryption) can also be used.

Although several preferred embodiments of this invention have been described in detail herein with reference to the accompanying drawings, it is to be understood that the invention is not limited to these precise embodiments, and that various changes and modifications may be effected therein by one skilled in the art without departing from the scope of spirit of the invention as defined in the appended claims.

What is claimed is:

1. A method of predicting whether an organic compound will penetrate the blood brain barrier significantly, the method comprising:
(a) determining whether the compound has fewer than six hydrogen bond donors and hydrogen bond acceptors; and
(b) based on whether the compound has fewer than six hydrogen bond donors and hydrogen bond acceptors, predicting whether the compound will penetrate the blood brain barrier significantly, wherein the compound is predicted to penetrate the blood brain barrier significantly only if it has fewer than six hydrogen bond donors and hydrogen bond acceptors.

2. The method of claim 1, wherein the method is implemented on a computing device.

3. The method of claim 2, further comprising:
receiving a representation of the compound; and
analyzing the compound in a manner that automatically identifies hydrogen bond donors and hydrogen bond acceptors.

4. The method of claim 2, further comprising redesigning the compound by adding one or more hydrogen bond donors and/or hydrogen bond acceptors, wherein the compound originally had fewer than six total hydrogen bond donors and hydrogen bond acceptors and now has six or more total hydrogen bond donors and hydrogen bond acceptors.

5. The method of claim 2, further comprising redesigning the compound by removing one or more hydrogen bond donors and/or hydrogen bond acceptors wherein the compound originally had six or more total hydrogen bond donors and hydrogen bond acceptors and now has fewer than six total hydrogen bond donors and hydrogen bond acceptors.

6. The method of claim 2, further comprising automatically repeating (a) and (b) for multiple different compounds.

7. A computer program product comprising a machine-readable medium on which are provided program instructions for predicting whether an organic compound will penetrate the blood brain barrier significantly, the instructions comprising:
(a) determining whether the compound has fewer than six hydrogen bond donors and hydrogen bond acceptors; and
(b) based on whether the compound has fewer than six hydrogen bond donors and hydrogen bond acceptors, predicting whether the compound will penetrate the blood brain barrier significantly, wherein the compound is predicted to penetrate the blood brain barrier significantly only if it has fewer than six hydrogen bond donors and hydrogen bond acceptors.

8. The computer program product of claim 7, wherein the program instructions further comprise:
receiving a representation of the compound; and analyzing the compound in a manner that automatically identifies hydrogen bond donors and hydrogen bond acceptors.

9. The computer program product of claim 7, wherein the program instructions further comprise:
redesigning the compound by adding one or more hydrogen bond donors and/or hydrogen bond acceptors, wherein the compound originally had fewer than six total hydrogen bond donors and hydrogen bond acceptors and now has six or more total hydrogen bond donors and hydrogen bond acceptors.

10. The computer program product of claim 7, wherein the program instructions comprise:
redesigning the compound by removing one or more hydrogen bond donors and/or hydrogen bond acceptors wherein the compound originally had six or more total hydrogen bond donors and hydrogen bond acceptors and now has fewer than six total hydrogen bond donors and hydrogen bond acceptors.

11. The computer program product of claim 7, further comprising program instructions for automatically repeating (a) and (b) for multiple different compounds.

12. An apparatus for predicting whether an organic compound will penetrate the blood brain barrier significantly, the apparatus comprising:
(a) an interface for receiving representations of organic compounds;
(b) a memory coupled for communication with said interface; and
(c) a processor coupled for communication with said memory, wherein at least one of the memory and the processor includes logic for: (i) determining whether the compound has fewer than six hydrogen bond donors and hydrogen bond acceptors; and (ii) based on whether the compound has fewer than six hydrogen bond donors and hydrogen bond acceptors, predicting whether the compound will penetrate the blood brain barrier significantly, wherein the compound is predicted to penetrate the blood brain barrier significantly only if it has fewer than six hydrogen bond donors and hydrogen bond acceptors.

13. A method of analyzing a compound, the method comprising:
(a) determining a total number of hydrogen bond donors and hydrogen bond acceptors on the compound;
(b) determining a partitioning property of the compound;
(c) based on at least the total number of hydrogen bond donors and hydrogen bond acceptors, classifying the compound based upon its ability to penetrate the blood brain barrier; and
(d) based upon the total number of hydrogen bond donors and hydrogen bond acceptors and the partitioning property, classifying the compound according to at least one of (i) its solubility and (ii) its ability to be absorbed in the intestine.

14. The method of claim 13, wherein the compound is classified based on both its solubility and its ability to be absorbed in the intestine.

15. The method of claim 13, wherein the classifying of (d) is performed by determining where the compound lies with respect to a line or curve defining a transition between solubility/insolubility or absorption/non-absorption in the intestine, wherein the line or curve bisects a two-dimensional space of hydrogen bond donor and acceptor count versus partitioning property.

16. The method of claim 13, wherein (c) and (d) employ a model comprising:
a two-dimensional space of hydrogen bond donor and acceptor count versus partitioning property, and
a first line through said two-dimensional space, which first line separates a first region containing compounds that substantially penetrate the blood brain barrier from a second region containing compounds that do not substantially penetrate the blood brain barrier, and wherein the first line is substantially perpendicular to an axis specifying the total number of hydrogen bond donors and hydrogen bond acceptors in the two-dimensional space.

17. The method of claim 16, wherein the first line crosses the axis at a value of about six total hydrogen bond donors and hydrogen bond acceptors.

18. The method of claim 13, wherein the method is implemented on a computer.

19. A computer program product comprising a machine readable medium on which is provided program instructions for analyzing compounds, the program instructions specifying at least the following operations:
(a) determining a total number of hydrogen bond donors and hydrogen bond acceptors on a compound;
(b) determining a partitioning property of the compound;
(c) based on at least the total number of hydrogen bond donors and hydrogen bond acceptors, classifying the compound based upon its ability to cross the blood brain barrier; and
(d) based upon the total number of hydrogen bond donors and hydrogen bond acceptors and the partitioning property, classifying the compound according to at least one of (i) its solubility and (ii) its ability to be absorbed in the intestine.

20. The computer product of claim 19, wherein the program instructions specifying (d) classifies the compound based on both its solubility and its ability to be absorbed in the intestine.

21. The computer program product of claim 19, wherein the classifying of (d) is performed by determining where the compound lies with respect to a line or curve defining a transition between solubility/insolubility or absorption/non-absorption in the intestine, wherein the line or curve bisects a two-dimensional space of hydrogen bond donor and acceptor count versus partitioning property.

22. The computer program product of claim 19, wherein (c) and (d) employ a model comprising:
a two-dimensional space of hydrogen bond donor and acceptor count versus partitioning property, and
a first line through said two-dimensional space, which first line separates a first region containing compounds that substantially penetrate the blood brain barrier from a second region containing compounds that do not substantially penetrate the blood brain barrier, and wherein the first line is substantially perpendicular to an axis specifying the total number of hydrogen bond donors and hydrogen bond acceptors in the two-dimensional space.

23. The computer program product of claim 22, wherein the first line crosses the axis at a value of about six total hydrogen bond donors and hydrogen bond acceptors.

24. A machine readable medium on which is provided at least a representation of a two-dimensional space defined by a first axis over which a partitioning property of chemical compounds varies and a second axis over which the sum of hydrogen bond donors and hydrogen bond acceptors of chemical compounds varies,
wherein the two-dimensional space is divided into a first region defining compounds predicted to significantly cross the blood brain barrier and a second region defining compounds predicted to not significantly cross the blood brain barrier and a second region defining compounds predicted to not significantly cross the blood brain barrier and a second region defining compounds predicted to not significantly cross the blood brain barrier.

25. The machine-readable medium of claim 24, wherein the first and second regions are separated by a first line or curve that is substantially perpendicular to the second axis.

26. The machine readable medium of claim 25, wherein the first line crosses the second axis at about a value of six hydrogen bond donors and hydrogen bond acceptors.

27. The machine readable medium of claim 24, wherein the two-dimensional space is further divided into a third region defining compounds predicted to have a solubility greater than a defined value and a fourth region defining compounds predicted to have a solubility lower than the defined value, wherein the third and fourth regions overlap the first and second regions.

28. The machine readable medium of claim 27, wherein the two-dimensional space is further divided into a fifth region defining compounds predicted to have an absorption in the intestine of greater than a second defined value and a sixth region defining compound predicted to have an absorption in intestine of less than the second defined value, wherein the first and second regions overlap the fifth region.

29. The machine readable medium of claim 27, wherein the third region comprises a first sub-region defining compounds predicted to have a solubility of less than a second predefined value and a second sub-region defining compounds predicted to have a solubility greater than the second defined value.

30. The machine readable medium of claim 24, wherein the two-dimensional space is further divided into a third region defining compounds predicted to have an absorption in the intestine of greater than a defined value and a fourth region defining compounds predicted to have an absorption in the intestine of less than the defined value, wherein the third region overlaps with the first and second regions.

31. The machine readable medium of claim 30, wherein the third region comprises a first sub-region defining compounds predicted to have an absorption in the intestine of less than a second defined value and a second sub-region defining compounds predicted to have an absorption in the intestine of greater than the second defined value.

32. An apparatus for analyzing compounds to predict one or more medicinal properties, the apparatus comprising:
(a) an interface for receiving representations of chemical compounds;
(b) a memory coupled for communication with the interface; and
(c) a processor coupled for communication with said memory, wherein at least one of the memory and the processor contains logic for determining where a given compound resides in a two-dimensional space,
wherein the two-dimensional space is defined by a first axis over which a partitioning property of chemical compounds varies and a second axis over which the sum of hydrogen bond donors and hydrogen bond acceptors of chemical compounds varies, and
wherein a representation of the two-dimensional space employed by the apparatus is divided into a first region defining compounds predicted to significantly cross the blood brain barrier and second region defining compounds predicted to not significantly cross the blood brain barrier.

33. The apparatus of claim 32, wherein the first and second regions are separated by a first line or curve that is substantially perpendicular to the second axis.

34. The computer readable medium of claim 33, wherein the first line crosses the second axis at about a value of six hydrogen bond donors and hydrogen bond acceptors.

35. The apparatus of claim 32, wherein the two-dimensional space is further divided into a third region defining compounds predicted to have a solubility greater than a defined value and a fourth region defining compounds predicted to have a solubility lower than the defined value, wherein the third and fourth regions overlap the first and second regions.

36. The apparatus of claim 35, wherein the two-dimensional space is further divided into a fifth region defining compounds predicted to have an absorption in the intestine of greater than a second defined value and a sixth region defining compound predicted to have an absorption in intestine of less than the second defined value, wherein the first and second regions overlap the fifth region.

37. The apparatus of claim 35, wherein the third region comprises a first sub-region defining compounds predicted to have a solubility of less than a second predefined value and a second sub-region defining compounds predicted to have a solubility greater than the second defined value.

38. The apparatus of claim 32, wherein the two-dimensional space is further divided into a third region defining compounds predicted to have an absorption in the intestine of greater than a defined value and a fourth region defining compounds predicted to have an absorption in the intestine of less than the defined value, wherein the third region overlaps with the first and second regions.

39. The apparatus of claim 38, wherein the third region comprises a first sub-region defining compounds predicted to have an absorption in the intestine of less than a second defined value and a second sub-region defining compounds predicted to have an absorption in the intestine of greater than the second defined value.
